(19) United States
(12) Patent Application Publication
(10) Pub. No.: US 2020/0343116 A1
(43) Pub. Date: Oct. 29, 2020

(54) MOBILE INSPECTION SYSTEM FOR THE DETECTION OF DEFECT OCCURRENCE AND LOCATION

(71) Applicant: Matan LAPIDOT, Kfar Menachem (IL)

(72) Inventor: Matan LAPIDOT, Kfar Menachem (IL)

(21) Appl. No.: 16/336,486

(22) PCT Filed: Oct. 19, 2017
(86) PCT No.: PCT/IL2017/051153

§ 371 Date: Mar. 26, 2019

Related U.S. Application Data
(60) Provisional application No. 62/411,541, filed on Oct. 22, 2016.

(51) Int. Cl. H01L 21/67; G01N 33/00; G01N 21/95; G01N 27/20; G01N 15/10

(52) U.S. Cl. CPC H01L 21/67288; G01N 33/00; G01N 2033/0095; G01N 27/20; G01N 15/10; G01N 21/9501

(57) ABSTRACT

A system for the detection of defect occurrence and location in a fabrication process tool, which comprises an inspection wafer comprising a plurality of sensors and a power source, the inspection wafer configured to be inserted to the fabrication process tool and inspect the fabrication process tool; and a processing unit configured to receive as input data from the sensors and calculate location, time occurrences and physical characteristics of defects by comparing between data received from at least one of the sensors at different times throughout inspection of the fabrication process tool. The inspection wafer may further comprise one or more transmitters configured to transmit signals such that the sensors are able to detect changes in one or more properties of the signals that occur as a result of a defect. The inspection wafer may also comprise a logic device, a processing element and a memory device, where the logic device samples outputs of the sensors, the processing element processes the sampled sensor's output and stores the processed data on the memory device.

### FIELD OF THE INVENTION

The present invention relates to the field of semiconductor fabrication control. More particularly, the invention relates to a system for inspecting semiconductor fabrication process tools, and specifically detecting occurrences and locations of defects in the process tools.

### BACKGROUND OF THE INVENTION

In the modern era, Integrated Circuit (IC) fabrication is undergoing a continuous downsizing. Due to the complexity of the ICs and precision requirements of the fabrication processes, the importance of tight process control is constantly growing. Today, engineers in manufacturing and fabrication facilities of VLSI devices put in vast amounts of time and resources for process control and defect reduction in the processes. A fabrication process typically comprises (and is referred to herein as) operations, measurement, and tools (but not limited to those) used during fabrication of semiconductor devices, e.g. Very Large Scale Integration (VLSI) fabrication.

A defect in electronic manufacturing is defined as an undesirable change in the physical properties (e.g. mass, size or figure), the electrical, optical and/or mechanical properties or features of a wafer undergoing a fabrication process at a specific location of a wafer and at a specific time during of the process. For instance, a deposit layer thickness of 15 nanometer, instead of 12 nanometer, is considered as a defect. Another exemplary defect is a particle-coming in contact with the wafer at a specific location and time. Such foreign particles may destroy the manufactured die (a small block of semiconducting material, on which a given functional circuit is fabricated), particularly when the fabricated die includes very high density of electronic devices. In such dense environment, even very small particles may destroy the manufactured die.

During the manufacturing process of a wafer, the wafer is coupled to a wafer support chuck using vacuum or electrostatic force. Normally, it is essential that the wafer will be uniformly coupled to the chuck in order to avoid deformation and to assure smooth and uniform contact between the wafer backside and the chuck. Such a uniform contact is important to accurately control the temperature of the wafer during different manufacturing stages and to assure a uniform focal plane for precise lithography on the wafer's up-side layers. Any deformation will deteriorate the uniformity of heat transfer between the wafer backside and the surface of the chuck, as well as the uniformity of the focal plane. In addition back side particles might harm some robotics pads vacuum and might cause the wafer to be slippery, due to loose vacuum. Also, back side particles and other machinery defects can create scratches and cracks which later might cause wafer breakage events, due to thermal shock and/or different types of mechanical vibrations.

During the manufacturing process, foreign particles may land on the backside of a wafer. In response to an applied coupling force, even a relatively small particle (in the order of 1 μm) may cause wafer deformation that will deteriorate the wafer production process.

Particles can be present in a process as result of, for instance, one of the following particles may fall from a showerhead or from the chamber walls, a leak from the atmosphere into the vacuum, vibrations during robot motion, or even mechanical contact with the wafer.

Several inspection methods for detecting and reducing defects are commonly used in the VLSI industry. For instance, some factories use external standalone metrology tools that measure the process impact on the production wafer or a test wafer, and measure defects characteristic and location. However, in spite of its popularity, this method is far from providing a full picture inasmuch as no information may be obtained regarding the time occurrence of defect formations while processed inside the specific processing tool or in between the various tools while the wafer is being transferred between the tools and inside/outside of them. Moreover, the defect may originate from the metrology tool itself.

Other inspection methods comprise integrating metrology systems into process tools, thereby detecting the creation of a defect near or on a wafer in real time. These systems have visibility only for a partial path of the wafer motion throughout the process tool, since at least part of the integrated metrology system is stationary and limited in following the wafer path.

Another common inspection method is using autonomous inspection wafers, which currently allow defect formation detection in real time, however lack the ability to provide defects location on the wafer.

U.S. Pat. No. 5,274,434 discloses particle inspection method and apparatus for preventing occurrence of large quantities of defects and for keeping a necessary yield. The inspection apparatus is made up in a small-sized apparatus and disposed at inlet/outlet of processing apparatuses of the production line or to a transfer system between the processing apparatuses. The inspection apparatus includes a monitor for real-time sampling foreign particles possible deposited on wafer which is being carried by the transfer system, thereby enabling simplification in construction of the production line and reduction of manufacturing cost. The inspection apparatus may comprise a refractive index changeable type lens array, a spatial filter and a pattern data elimination circuit, and makes possible to conduct foreign particle inspection on repetitively-patterned portions of the wafers during transfer. With the spatial filter for eliminating repetitive data of the repetition patterns the small-sized compact inspection apparatus is capable of real-time inspecting the foreign particles on the wafers at a high speed. However, the inspection apparatus is stationary and is not able to determine the timing of a deposited particle.

US 2014/0208850 discloses a semiconductor device defect detecting apparatus, which includes a sensor disposed on semiconductor process equipment. The sensor is configured to detect a signal emitted from a semiconductor device in contact with the semiconductor process equipment and a signal analyzer configured to determine whether the semiconductor device is defective based on the detected signal in a predetermined frequency range.

U.S. Pat. No. 6,966,235 discloses remote sensors for monitoring of process parameters on the surface, sub-surface, or surrounding environment of manufactured semiconductor substrates. The remote sensors are attached directly to the product material, allow for nonintrusive entry into the manufacturing area, via the same robotic handling or automated systems used to transport the standard product material. Data is recorded from the sensors, by wireless transmission, or when a signal is impassible, data is recorded in an on-board memory, which stores the data for later downloading. However, these remote sensors are directed to detect parameters such as die thickness, uniformity and are incapable of detecting foreign particles in size of few nanometers.

All existing methods fail to provide information about both the location and time of defect formations in general, and that of particle type of defect in particular. This information is obviously vital for an optimal fabrication process in at least two aspects:
1. It provides information regarding the orderliness of the process tool, enabling to:
   a. Anticipate tools malfunctions before they actually occur; and
   b. Provide tool operator better information regarding the process tool performance, in order to optimize production yield in terms of process quality;
2. This information may significantly allow shortening the time required for locating the source of particles inside a process tool which has been identified as malfunctioned, thereby saving “down-time” and increasing overall production yield.

It is therefore an object of the present invention to provide a system for detecting defect formations in semiconductor fabrication processes and the location and time thereof.

It is another object of the present invention to provide a system for detecting defect formations in semiconductor fabrication processes, which is mobile.

It is another object of the present invention to provide a system for detecting defect formations in semiconductor fabrication processes, which emulates a real wafer during manufacturing process.

It is a further object of the present invention to provide a system for detecting foreign particles becoming in contact with the backside of a wafer during semiconductor fabrication processes.

Other objects and advantages of the invention will become apparent as the description proceeds.

### SUMMARY OF THE INVENTION

The present invention is directed to a system for the detection of defect occurrence and location in a fabrication (e.g., process, metrology, alignment or storage) tool, which comprises:
a. an inspection wafer comprising a plurality of sensors and a power source, the inspection wafer configured to be inserted to the fabrication process tool and inspect the fabrication process tool; and
b. a processing unit (such as a computed station remote from the inspection wafer) configured to receive as input data from the sensors and calculate location (on the inspection wafer), time occurrences and physical characteristics of defects by comparing between data received from sensors at different times and/or/at different locations (on the inspection wafer) throughout inspection of the fabrication tool.

The system is adapted to detect presence of a particle in the fabrication process, which may be selected from the group of:
dielectric particles;
metallic particles;
semi-conducting particles;
Organic particles;
particles originating from within the process tool;
particles originating from materials flowing inside the tool; and
particles originating from outside the process tool.

The inspection wafer may further comprise one or more transmitters configured to transmit signals such that the sensors are able to detect changes in one or more properties of the signals that occur as a result of a defect.

The inspection wafer may further comprise a logic device, a processing element and a memory device, wherein the logic device samples outputs of the sensors, the processing element processes the sampled sensor's output and stores the processed data on the memory device.

The inspection wafer may further comprise communication elements configured to transmit data to the remote computed station.

The sensors are selected from a list comprising:
one or more capacitor sensors;
one or more photocathodes;
one or more photo detector sensors;
one or more Micro electro mechanical (MEM) devices;
one or more capacitors micro machined ultrasonic transducer;
one or more oscillator devices configured to measure energy or mass changes;
A resonance electro/optical device
one or more pressure sensors;
one or more temperature sensors; or
Quartz Crystal Micro balance sensors; or
a combination of two or more of the above.

The resistivity of the sensors may be measured according to Van Der Pauw resistivity method.

One or more of the sensors may comprise piezoelectric materials and piezoelectric components, or a dielectric waveguide in contact with a metallic layer or a metallic pattern suitable to generate a Plasmonic reaction.

One or more transmitters may be selected from a list comprising the following:
one or more light emitting devices;
one or more electron beam sources;
one or more ultrasonic source; or
a combination of two or more of the above.

An object and/or a particle on a surface of the inspection wafer may be detected by back scatter technique.

The physical characteristics may be selected from the group of:
Size;
Shape;
Mass;
Conductivity;
capacitance
Dielectric properties;
Refractive index.

The system may further comprise a protective layer (made e.g., dielectric layer, etch stop later, etc.), for protecting a wafer from wafer-modifying processes.

The system may further comprise a docking station for:
charging the power source;
wafer cleaning;
wafer re-coating;
Configuring the inspection plan.

The power supply may be selected from the group of:
A monolithic power supply;
a hybrid power supply;
a capacitor;
a battery.

The optical sensors may be selected from the group of:
Optical resonators;
micro-ring resonators;
photonic crystal structure resonators;
Plasmonic Crystal structure resonators.

The resonance wavelength may be affected by the presence of a defect.

The resonance wavelength may be detected by change of amplitude or phase in a wavelength-specific detector/transmitter.

The processing and memory resources may be reduced using a common receiver for multiple sensors cells array.

In one aspect, a minimal emittance signal is provided to the receiver under normal conditions, when there is no presence of a defect.

Minimal emittance may be achieved by a plasmonic/non-plasmonic grating structure on top of a wave-guide, to create destructive interferences of the plasmonic and/or photonic waves.

### BRIEF DESCRIPTION OF THE DRAWINGS

In the drawings:

FIG. 1 schematically illustrates a side view of an inspection wafer according to an embodiment of the invention;

FIG. 2 schematically illustrates a side view of an inspection wafer according to another embodiment of the present invention; and

FIG. 3a schematically illustrates a side view of an inspection wafer according to yet another embodiment of the present invention;

FIG. 3b illustrates a hierarchical implementation of the memory/readout circuit with reduced computing resources (memory and processing power), according to an embodiment of the invention;

FIG. 4 is a top view of an inspection wafer system 300, according to an embodiment of the invention; and

FIGS. 5-6 schematically illustrate a side view of an inspection wafer which is capable of detecting backside particles, according to embodiments of the present invention.

### DETAILED DESCRIPTION OF THE INVENTION

Reference will now be made to an embodiment of the present invention, examples of which are illustrated in the accompanying figures for purposes of illustration only.

One skilled in the art will readily recognize from the following description that alternative embodiments of the structures and methods illustrated herein may be employed, mutatis mutandis, without departing from the principles of the claimed invention.

The present invention provides a system for inspecting a fabrication process during a full or partial wafer path from the carrier box to the tool and back to the carrier box or while the boxes are transferred between tools, due to particle-contamination inside the boxes. The system comprises an array of sensors integrated into a device (hereinafter an inspection wafer) by a semiconductor fabrication process (e.g. VLSI), the dimensions of which imitate those of a standard size wafer used in the semiconductor manufacturing process being inspected. The amount, density, type (affecting defect-material sensitivity and defect-size sensitivity) and distribution of sensors on an inspection wafer are defined by the process needs for a given operation (e.g. etch, ultrasonic clean, CMP etc.).

The sensors of the inspection wafer allow detection of a defect location across the wafer in addition to the location of defect formation along the path traveled by the wafer as it passes throughout the process. Furthermore the sensors allow detecting the time of a defect formation along the wafer path inside a desired process tool (for example: Chemical vapor deposition (CVD), Physical vapor deposition (PVD), Photolithography, Metrology tools etc.) thereby indicating the position inside the process tool responsible for defects formation by the tool. For example, the system can tell that a defect was created on the wafer center or edge through the buffer chamber. The outputs of the sensors are processed by processing elements, provided with the system, which determine if, where and when a defect occurred as well as provide defect characteristics (e.g. mass, physical size and/or shape, electrical conductivity, light transmission and/or reflection and/or scattering characteristics, electrical charge, electrical capacitance, etc.).

It is noted that although defect detection is demonstrated throughout the description by detection of a particle, the present invention isn't limited to detecting a specific defect, and can also be used for detecting, for instance scratches, an extra pattern or a missing pattern (used as an identifying signature of a specific fabrication tool).

According to an embodiment of the present invention, the inspection wafer can also inspect the fabrication process with the aid of an external processing unit that receives and processes the data collected by the inspection wafer for running further analyses in order to get high resolution and considerable information regarding the detected defects.

According to another embodiment of the present invention, a portion of the data processing is performed by processing elements integrated into the inspection wafer itself (e.g. by semiconductor fabrication). Use of integrated processing elements is suitable for filtering noise or unnecessary data from being sampled and stored on the memory device.

After inspection by the sensors throughout a fabrication process, possibly pre-processing them, and storing the sensors data, a computer software program (running on a processing unit such as a remote computed station) receives the sensors data (possibly after being processed as described above) as input and characterizes the defects in all abovementioned aspects. This may be achieved by comparing between the results of a single sensor or an array of sensors at different times. For example, a first measurement of a first group of sensors may be compared to a second measurement of the same group of sensors, i.e. at another time, or to a measurement of another group of sensors (either measured at the same time as the first measurement or at another time).

According to another embodiment of the present invention, the inspection wafer transmits (on wafer) pre-processed sensors data via wired/wireless communication (e.g. RF, Bluetooth etc.) to a processing unit such as a remote computed station where the data is collected on, for post-processing and analysis (outside the wafer). According to another embodiment of the invention, the remote station charges the portable wafer. According to another embodiment of the invention, the remote station is used for configuring various parameters of the inspection wafer (e.g. sensor illumination intensity, sensor sensitivity, degree of sensors binning, etc.).

The inspection wafer may comprise one or more types of sensors, wherein the sensor types are determined according to the inspection and detection requirements of a given process under inspection. For instance, an inspection wafer may comprise at least one of, or a combination of, the following:
one or more electrical capacitance sensors (e.g. CMOS capacitance sensor and/or RC sensors);
one or more electrical resistance sensor
one or more photocathodes;
One or more photo detector sensors (e.g. CCD, CMOS, PN junction sensor, etc.).
one or more capacitors micro machined ultrasonic transducer;
one or more oscillator devices configured to measure energy or mass changes (e.g. micro electromechanical MEM device);
one or more resonance electro/optical devices (e.g. ring resonator);
one or more plasmonic devices;
one or more photonic-crystal devices (e.g. photonic crystal waveguide)
one or more pressure sensors; and/or
one or more temperature sensors.

The resistivity of the sensors can be measured based on Van Der Pauw resistivity method (a technique commonly used to measure the resistivity and the Hall coefficient of a sample of any arbitrary shape, so long as the sample is approximately two-dimensional, solid (no holes) and the electrodes are placed on its perimeter) or any other electrical resistivity method known in the art. According to an embodiment of the invention, the sensors comprise piezoelectric materials and piezoelectric components, e.g. Quartz Crystal micro balance.

According to another embodiment of the invention, one or more sensors comprise a dielectric waveguide covered by a metallic layer or a metallic pattern suitable to generate a Plasmonic excitation and/or plasmon-polariton excitation. Plasmonic sensors are well known in the art, and an exemplary description and implementation can be found in Nanostructured Plasmonic Sensors by Matthew E. Stewart et. al, Department of Chemistry, University of Illinois at Urbana-Champaign, Urbana, Ill. 61801, Department of Materials Science and Engineering, University of Illinois at Urbana-Champaign, Urbana, Ill. 61801, and Chemistry Division and Center for Nanoscale Materials, Argonne National Laboratory, Argonne, Ill. 60439. When a Plasmon wave interacts with an airborne particle the output of the wave guide changes. This can be detected using an electro-optical device such as a photo diode or a spectrometer.

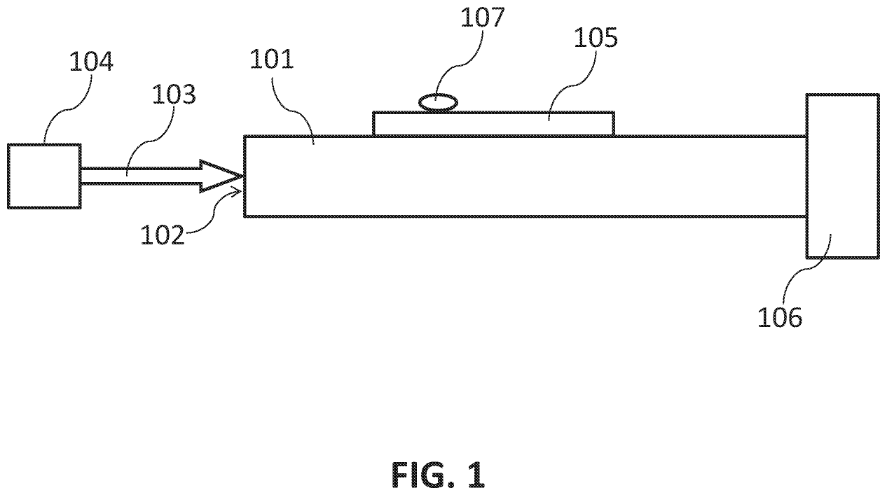

FIG. 1 schematically illustrates a side view of an inspection wafer according to an embodiment of the invention, comprising a waveguide 101 with an input 102 to which a signal 103 from a transmitter 104 is input, a protection layer 105 (such as a plasmonic layer made of plasmonic metamaterial that uses surface plasmons to achieve optical properties not seen in nature. Plasmons are produced from the interaction of light with metal-dielectric materials) on the wafer's top side, and a detection component 106 at the output configured to measure the signal at the output. According to changes in the measured signal caused by a defect 107, the system is capable of detecting defects in the process, depending on relative location of the sensor on the inspection wafer and the time of the detection (thereby revealing the location along the path traveled by the wafer inside the process tool in which the defect was formed on the wafer).

According an embodiment of the present invention, the detection layer of the inspection wafer includes Micro-Electro-Mechanical devices (MEMs) array and/or capacitors micro machined ultrasonic transducers and/or oscillator device which can measure energy or mass changes. According to still another embodiment, the arrays of sensors comprise pressure and/or temperature sensors.

According to an embodiment of the invention, the transmitter 103 comprises electron beam sources, ultrasonic sources, light emitting devices in various wavelengths, e.g. LED or laser diode, or any other component capable of producing a signal whose properties are affected by the presence of a particle 107. The transmitted signal will transmit through a protection layer 104 that comprises dielectric or another material that allows transmitting towards a defect or particles 107 on the top of the surface. The particle 107 on top of layer 104 will scatter the signal back to the sensors 105. The protection layer 104 can be coated with anti-reflected layer or another optic layer. According to another embodiment of the invention, the array of sensors comprises different stacks of optical layers that impact the transmission, the reflection, the absorption and the phase of the emitted and reflective signals. An additional layer may be provided for protecting the sensors and detection layer. According to an embodiment of the invention, the transmitter (e.g. a LED or laser diode) is fabricated to the inspection wafer.

The inspection wafer further comprises at least one portable or stationary power source (e.g. one or more batteries, not shown in the figures), providing AC and/or DC electrical power to all electronic components and sensors and any other component on the inspection wafer requiring electrical power for their operation. According to an embodiment of the invention, the power source comprises capacitors or super-capacitors that are fabricated to the inspection wafer.

The inspection wafer further comprises a time based circuit suitable to sample the output signal from the sensors, in addition to an analog to digital convertor for converting the sampled analog signal to a digital representation thereof for allowing computer processing of the signal.

According to an embodiment of the present invention, a back scatter technique is used for detecting objects and/or particles on the surface of a substrate/wafer and/or on the surface of a deposit layer of a substrate/wafer.

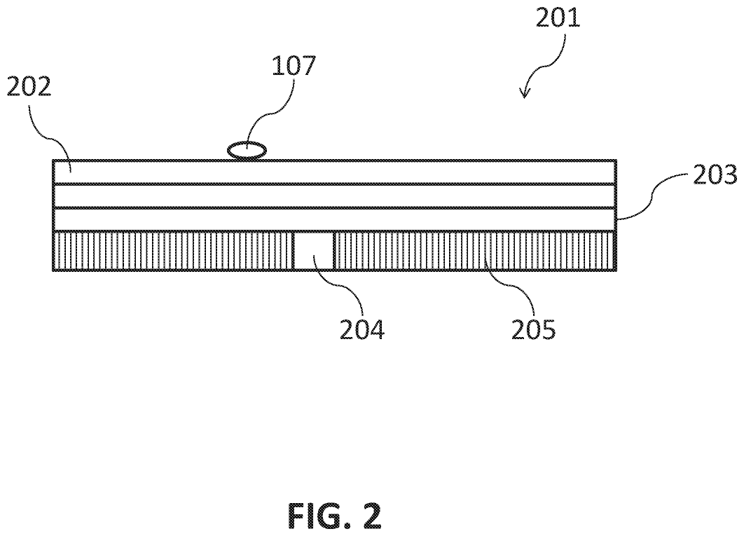

FIG. 2 schematic illustrates (side view) the structure of a basic back scattered cell of a detection sensor, according to an embodiment of the invention. The side view shows a basic sensor single cell 201, which is composed from an emitter (for example LED) 204, which emits the light toward the surface of the protection layer 202. In case of presence of a foreign particle, light will be scattered back to the collector, which consists of one or more collectors (i.e.: a photo diode or other sensor types) as illustrate by 205. Anti-reflective layer 203 keeps the noise from the collecting sensors as lower as possible.

According to an embodiment of the present invention, a back scattered technique is used for detecting defects in VLSI fabrication processes.

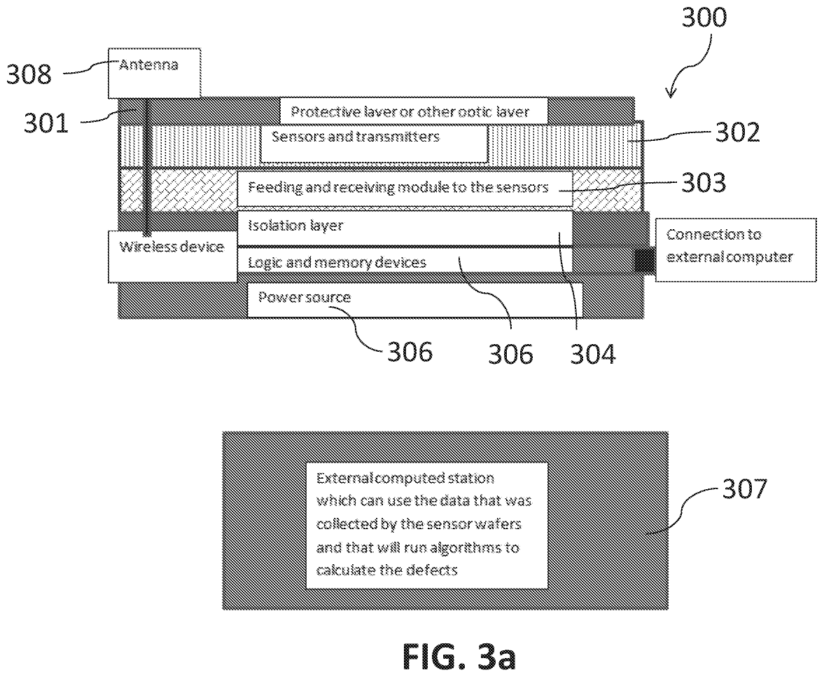

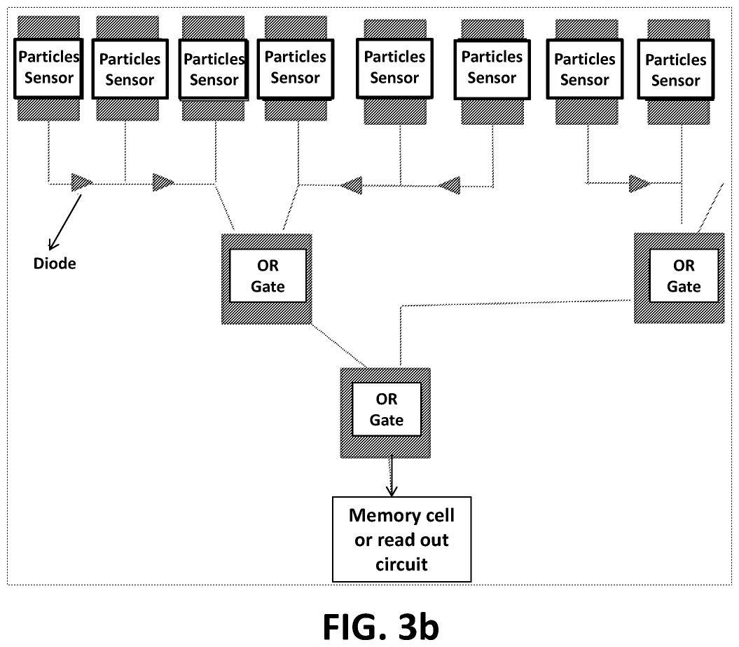

FIG. 3a schematically illustrates the structure of an entire inspection wafer system, according to an embodiment of the invention. The inspection wafer system 300 comprises a protective layer 301 or an anti-reflective layer, which is used to protect the sensors layer from mechanical, thermal, electrical and/or chemical damages. Protective layer 301 can be combined with or separated from the anti-reflective layer to reduce the reflection from the surface of the inspection wafer system.

The inspection wafer system 300 also comprises a sensors layer 302, which consists of collectors and emitters, to allow the detection of defects.

The inspection wafer system 300 also comprises a feeding and receiving module 303, to allow power feeding and/or the transmission of signals into the sensors. The feeding and receiving module 303 also comprises a readout circuit, to read out the signals from the sensors layer. An Isolation layer 104 isolates the microprocessors from electronic noise. Logic, computing and memory devices layer 305 is used to store and to compute the signals from the readout circuits.

The inspection wafer system 300 also comprises a power source 306 to supply power to the sensors layer 302, to the readout circuits, to the Logic, computing and memory devices layer 305 and to all other power consumption components that may be required.

An external computing station 307 is used to charge the power source 306, to perform additional computations from the data which was stored or transmitted during the wafer path inspection, to configure the inspection plan for the inspection wafer system 300 (i.e., to determine which sensors to use, to determine the sampling rate at each location and/or at each time, to determine the emitter power, etc.).

Antenna 308 is embedded to allow the inspection wafer 300 to autonomously communicate with external communication devices (such as external computing station 307).

Accordingly, one or more transmitters and a plurality of sensors are provided on a single plane or, in other embodiments of the invention, on a different plane. The sensors allow detecting the coordinate of a defect or a particle. The detection is output from the inspection wafer through readout circuits. The amount and type of sensors and transmitters, in addition to the configuration and density thereof are defined by the process needs and the given operation.

FIG. 3b illustrates a hierarchical implementation of the memory/readout circuit with reduced computing resources (memory and processing power), according to an embodiment of the invention. The spatial resolution (the ability to distinguished between two nearby particles between two adjacent particles) can be reduced, since there is no need for a spatial resolution in size orders of microns as long as the detection of a single nanometer scale particle is not compromised (i.e., the spatial resolution of the Mobile Inspection System (MIS) is compromised, but with minimal or no compromise of its detection-sensitivity). In order to reduce the amount of processing power and memory cells which are needed (for readout and compute all the readings), an effective cell which will consist of a defined amount of basic cells (emitter and receiver) with addition of applied MOS logic circuits can be used. For example, by applying an “OR” function between 10000 basic cells (of 10×10 micron) to give a binary or analog output when one or more basic cells in the effective cell applying current or voltage change which is greater than a certain threshold, the amount of the total readings at the system can be reduce by factor of 10000 to 7065000. In this case, the spatial resolution will be reduced for effective cells of 1 square millimeter while keeping the ability to detect a single Nano size scale particle.

The mount of readings may be further reduced using a multi-layer hierarchy, as shown in FIG. 3b. For example, the first layer will consist of an effective cell (using MOS logic) which will be constructed from 10K basic cells as explained before, then another layer with logic that can be applied to 10K effective cells to reduce the effective area to be 0.01 square cm and to reduce the amount of system readings by another factor of 100 to 70650. The multi-layer hierarchy with MOS logic circuits can be applied in with different number and levels of layers and with different logic.

Another possible solution to reduce processing and memory resources is to use a common receiver for multiple sensors cells array. In this case, a minimal signal will be provided to the receiver under normal conditions (when there is no presence of a defect). For example, in order to reduce the number of readouts and the computing power, a surface Plasmon resonance sensor arrays may be used to transmit minimal emittance, as long as there is no particle on top of is active area, by using one collector (receiver) for multiple sensors which are at the same adjacent area.

Minimal emittance can be achieved by a plasmonic grating (or non-plasmonic) structure on top of a wave-guide to create destructive interferences (base on Brag condition for destructive interference) of the plasmonic and/or photonic waves at the output of the waveguide or at the output of a photonic and/or plasmonic crystal.

This entire structure might be fabricated as a monolithic or a hybrid solution with the system. Minimal output signal mode can also be achieved using MOS types inverters at the output of the sensor.

The proposed multi-level hierarchy can be applied directly at the Silicon level by VLSI design and manufacturing.

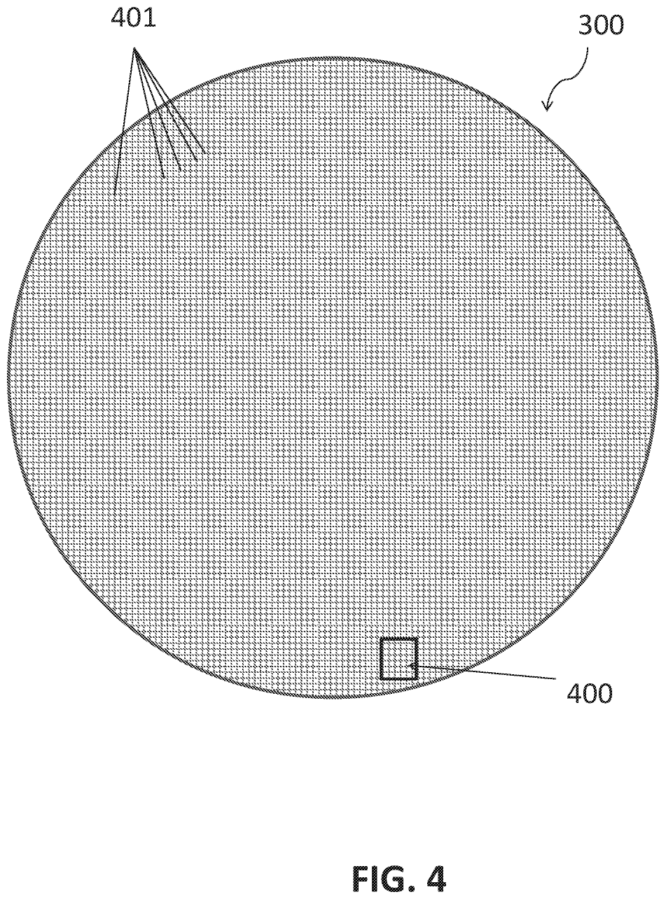

FIG. 4 is a top view of an inspection wafer system 300, according to an embodiment of the invention. It can be seen that the inspection wafer system comprised an array of dense basic cells (indicated by squares that cover the entire area) which may range between thousands and hundreds of thousands, depending on the required resolution. Reference numeral 401 shows an effective cell, which consists of a sub-array of basic cells which consists of a predetermined amount of basic cells (emitter and receiver) which are connected by MOS logic circuits, as illustrated in FIG. 3b above.

The inspection wafer system proposed by the present invention is also capable of detecting particles that landed on the backside of a wafer and may deteriorate the wafer production process.

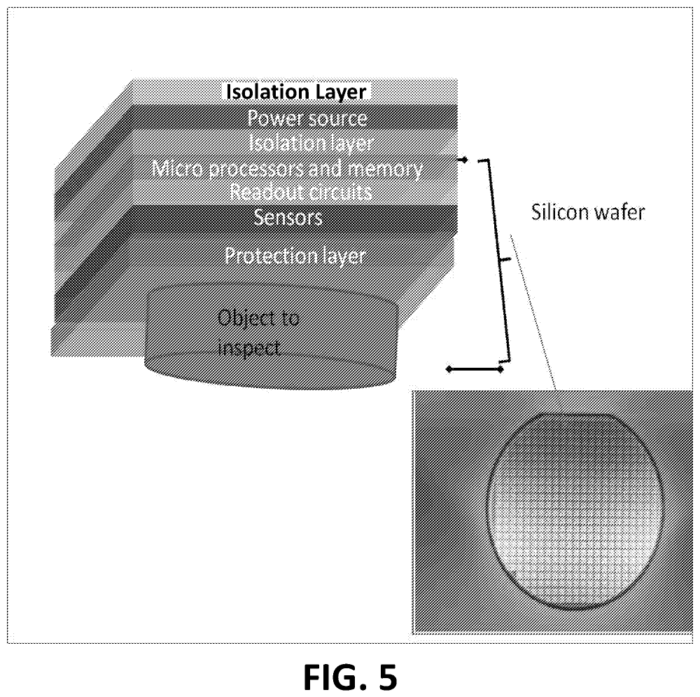

FIG. 5 schematically illustrates a side view of an inspection wafer which is capable of detecting backside particles, according to another embodiment of the present invention. In this embodiment, the mobile inspection system is mounted upside down.

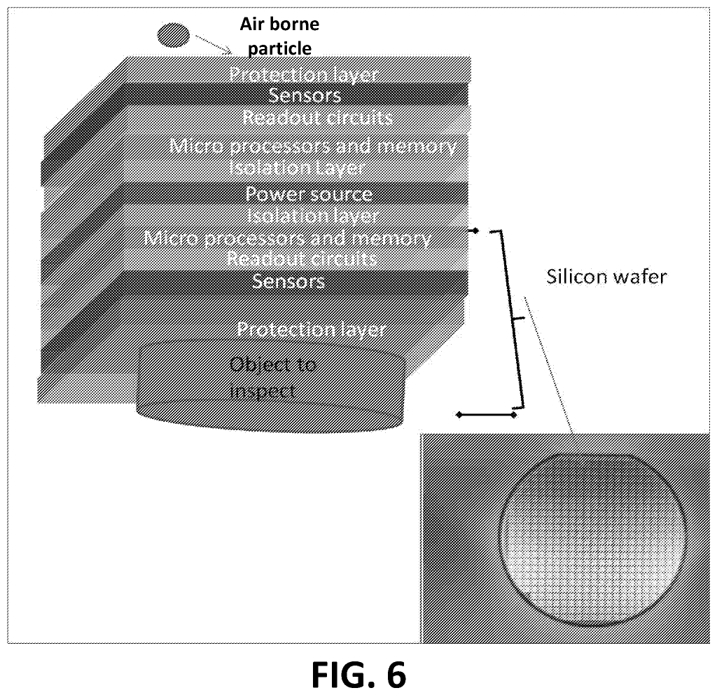

FIG. 6 schematically illustrates a side view of an inspection wafer which is capable of detecting backside particles, according to another embodiment of the present invention. In this embodiment, a sensors layer as well as readout electronics and/or any other layer is added to the backside of the MIS.

According to a first implementation, the added layers may be similar to the up-side layers using similar detection technology at the bottom of the Mobile Inspection System (MIS). According to another implementation, the added layers may be different from the up-side layers and may use different detection technology at the bottom of the Mobile Inspection System (MIS). Both implementations allow detecting particles or defects in both sides (front side and back side).

Particles detected according to the implementations illustrated in FIGS. 5-6 are not necessarily airborne, and may typically originate from the wafer-chuck/holder in the production tools.

Although embodiments of the invention have been described by way of illustration, it will be understood that the invention may be carried out with many variations, modifications, and adaptations, without exceeding the scope of the claims.

1. A system for the detection of defect occurrence and location in a fabrication process tool, comprising:
c. an inspection wafer comprising a plurality of sensors and a power source, the inspection wafer configured to be inserted to the fabrication process tool and inspect the fabrication process tool; and
d. A processing unit configured to receive as input data from the sensors and calculate location, time occurrences and physical characteristics of defects by comparing between data received from at least one of the sensors at different times throughout inspection of the fabrication process tool.

2. A system according to claim 1, adapted to detect presence of a particle in the fabrication process.

3. A system according to claim 2, adapted to detect presence of a particle selected from the group comprising:
dielectric particles;
metallic particles;
semi-conducting particles;
particles originating from within the process tool;
particles originating from materials flowing inside the tool; and
particles originating from outside the process tool.

4. A system according to claim 1, in which the inspection wafer further comprises one or more transmitters configured to transmit signals such that the sensors are able to detect changes in one or more properties of the signals that occur as a result of a defect.

5. A system according to claim 1, in which the inspection wafer further comprises a logic device, a processing element and a memory device, wherein the logic device samples outputs of the sensors, the processing element processes the sampled sensor's output and stores the processed data on the memory device.

6. A system according to claim 1, in which the processing unit is a computed station remote from the inspection wafer.

7. A system according to claim 5, in which the inspection wafer further comprises communication elements configured to transmit data to the remote computed station.

8. A system according to claim 1, in which the sensors are selected from a list comprising the following:
one or more electrical capacitor sensors;
one or more electrical resistance sensors;
one or more photocathodes;
one or more photo detector sensors;
one or more Micro electro mechanical (MEM) devices;
one or more capacitors micro machined ultrasonic transducer;
one or more oscillator devices configured to measure energy or mass changes;
A resonance electro/optical device
one or more pressure sensors;
one or more temperature sensors; or
a combination of two or more of the above.

9. A system according to claim 1, in which the resistivity of the sensors is measured according to Van Der Pauw resistivity method.

10. A system according to claim 1, in which the sensors comprise piezoelectric materials and piezoelectric components.

11. A system according to claim 1, in which one or more of the sensors comprise a dielectric waveguide in contact with a metallic layer or a metallic pattern suitable to generate a Plasmonic reaction.

12. A system according to claim 4, in which the one or more transmitters are selected from a list comprising the following:
one or more light emitting devices;
one or more electron beam sources;
one or more ultrasonic source; or
a combination of two or more of the above.

13. A system according to claim 4, in which an object and/or a particle on a surface of the inspection wafer is detected by back scatter technique.

14. A system according to claim 1, in which the physical characteristics are selected from the group of:
Size;
Shape;
Mass;
Conductivity;
capacitance.

15. A system according to claim 1, further comprising a protective layer, for protecting a wafer from wafer-modifying processes.

16. A system according to claim 1, further comprising a docking station for:
charging the power source;
wafer cleaning;
wafer re-coating.

17. A system according to claim 15, in which the protective layer is made of plasmonic metamaterial.

18. A system according to claim 1, in which the power supply is selected from the group of:
A monolithic power supply;
a hybrid power supply;
a capacitor;
a battery.

19. A system according to claim 8, in which the optical sensors are selected from the group of:
Optical resonators;
micro-ring resonators;
photonic crystal structure resonators.

20. A system according to claim 19, in which the resonance wavelength is affected by the presence of a defect.

21. A system according to claim 8, in which the resonance wavelength is detected by change of amplitude in a wavelength-specific detector/transmitter.

22. A system according to claim 5, in which processing and memory resources are reduced using a common receiver for multiple sensors cells array.

23. A system according to claim 22, in which a minimal emittance signal is provided to the receiver under normal conditions, when there is no presence of a defect.

24. A system according to claim 23, in which minimal emittance is achieved by a plasmonic/non-plasmonic grating structure on top of a wave-guide, to create destructive interferences of the plasmonic and/or photonic waves.
